(19) United States
(12) Patent Application Publication
(10) Pub. No.: US 2008/0058421 A1
(43) Pub. Date: Mar. 6, 2008

(54) COMPOSITIONS FOR CLEANING AND DISINFECTING NASAL TRACT AND SINUS CAVITY

(76) Inventor: John Alex Lopes, Troy, MI (US)

Correspondence Address: BROOKS KUSHMAN P.C., 1000 TOWN CENTER, TWENTY-SECOND FLOOR, SOUTHFIELD, MI, 48075, US

(21) Appl. No.: 11/764,950

(22) Filed: Jun. 19, 2007

Related U.S. Application Data
(60) Provisional application No. 60/841,962, filed on Sep. 5, 2006.

(51) Int. Cl. A61K 31/19; A61K 31/10; A61K 31/194

(52) U.S. Cl. 514/574; 514/557; 514/578; 514/710

(57) ABSTRACT

Biodegradable compositions for cleaning and disinfecting nasal tract and sinus cavity using food additive ingredients or ingredients that are safe.

### CROSS-REFERENCE TO RELATED APPLICATIONS

This application claims the benefit of U.S. provisional application Ser. No. 60/841,962 filed on Sep. 5, 2006.

### BACKGROUND OF THE INVENTION

1. Field of the Invention

This invention relates to biologically active microbicidal compositions for cleaning and disinfecting the nasal and sinus tract of the upper respiratory system.

2. Background Art

During normal breathing, environmental dust, mold, fungi, viruses, bacteria, pollens, chemical pollutants and other particulate matter are deposited in the nasal and sinus cavities. The inhaled particles can develop into infections, allergies and other maladies associated with the respiratory system.

Using a microbicidal composition to clean and disinfect the system with a nasal spray or inhaler offers early mediation and protection against the risk of subsequent onset of infections and other clinical manifestations associated with the inhaled matter.

Nasal inhalers or sprays have been used for mitigating allergic conditions, instilling antihistamines, and delivering mentholated sinus pain medications. Salt solutions also have been used for cleaning nasal and sinus cavities. Most salt solutions do not possess the microbicidal efficacy needed to be useful against infectious agents. Recently, vaccines have been instilled by the nasal route for immunizing purpose. However these remedies are used once the detrimental manifestations of inhaled matter have taken place. These remedies may come too late to thwart the harmful clinical process once underway. Early intervention is more economical for prevention of infections and allergic conditions by removing the causative agents either by cleaning or in the case of microbial agents by disinfecting. The use of antibiotics in the nasal microbicidal compositions is avoided due to the danger of developing allergies to the antibiotics and inciting microbial resistance to the antibiotic in the resident microbial population of the nasal and sinus cavities. Other antimicrobial agents such as alcohols, quaternary ammonium compounds, hydrogen peroxide, phenols can be considered toxic in nasal spray or inhalers.

### SUMMARY OF THE INVENTION

The present invention is based on the combined cleaning and microbicidal properties of ingredients in the compositions considered food additives or Generally Regarded As Safe (GRAS) by the United States Food and Drug Administration (USFDA). With food additives and/or GRAS ingredients, the disclosed nasal spray or inhaler will be safe and non-toxic for cleaning and disinfecting the nasal and sinus cavities of the upper respiratory tract.

The present invention teaches the preparation of a safe cleaning and microbicidal composition without the use of compounds that are toxic, corrosive or harmful. The present invention advances the art by introducing, in one aspect, a novel biologically active cleaning, disinfecting and sanitizing composition that is economical, useful for preventing respiratory associated infections and allergies in everyday life.

The present invention combines the broad-spectrum microbicidal or germ killing properties of food additive anionic surface-active agents and those of compounds that are Generally Regarded As Safe (GRAS) at pH values at or below 7.0. The anionic surface-active agents incorporated in the present invention serve both as cleaning agents as well as microbicidal agents. The anionic surface-active agents are commonly used in cleaning compositions. At low pH values, these surface-active agents are also used for the preparation of sanitizers or disinfecting solutions. For use in a nasal cleaning and disinfecting spray, the anionic surface-active agent must be present in an optimum amount.

Preferably, the invention has a concentration of anionic surface-active agents at low pH with optimum microbicidal properties. Medicinal emollients such as Aloe vera extract may be incorporated in the solution/cream without the loss of its microbicidal properties when the products are designed for use on the sinus and nasal mucosa. Other polymeric compounds (gums) and optional emollients that are stable at lower pH but do not neutralize the microbicidal properties can also be incorporated. The gums and polymeric ingredients can slowly release the ingredients and prolong the disinfecting activity of the compositions in the nasal and sinus cavities.

In use, the inventive compositions also eliminate the use of additional antimicrobial agents while delivering cleaning and microbicidal properties. Thus the present invention advances the art of preparing biologically active nasal and sinus cleaning and disinfecting compositions without the extra cost of additional ingredients. It also offers additional safety measures by eliminating the use of undesirable chemicals such as hydrogen peroxide, iodine, quarternary ammonium compounds and phenols.

The proposed biologically active cleaning and disinfecting nasal spray compositions incorporate enhanced microbicidal properties of either anionic surface-active agents or that of a mixture of organic acids at an acidic pH at or below neutrality. The FDA in the 21 Code of Federal Regulation lists these ingredients as either food additives or GRAS. The nasal route is used to deliver the biologically active cleaning and disinfecting solution by spray, mist, or as simply applying as a nostril cream.

### DETAILED DESCRIPTION OF THE PREFERRED EMBODIMENT(S)

In accordance with the invention, there is provided a class of chemical agents that can be used to prepare biologically active antimicrobial, sanitizing and/or disinfecting solutions or creams for cleaning nasal and/or sinus passages, as well as disinfecting nasal and/or sinus cavities of inhaled pollens, dust and chemical pollution particles, viruses, molds, fungi, bacteria and other allergic and infectious agents. The preparations can also be used to clean and disinfect other inanimate surfaces.
a. The biologically active cleaning and disinfecting antimicrobial spray/solution/cream composition essentially consists of one or more food additives or GRAS anionic surface-active agent or a mixture thereof,
b. Food grade organic or inorganic acids as acidifying agent to lower the pH at or below 7.0,
c. Water or a suitable solubilizing agent.

The biologically active composition may include other compatible ingredients, which do not reduce or interfere with the cleaning and disinfecting properties. The composition may additionally carry an emollient, a coloring agent, antioxidants, natural or synthetic medicinal ingredients, fragrances, vitamins, and moisturizing agents.
a. The anionic surface-active agent has the following general formula: R−A n−C n+wherein R is hydrophobic group consisting of substituted or unsubstituted n-alkyl, n-alkenyl, alkylbenzyl, or alkylnapthalene group with a length equivalent to 6 to 16 carbon atoms; wherein A is anionic group selected from monocarboxylic, dicarboxylic, sulfate, sulfonate, phosphate and phosphonate group or mixture thereof; wherein C is cationic group capable of forming electrostatic linkage with A. n represents a number of ionic charges. The anionic agents may be used in concentrations ranging from about 10 ppm to about up to 100,000 ppm. The anionic agent can be used as a single molecular species or in combination with other anionic molecular species. Other foam generating agents and surface tension reducing agents that help cleaning process may be incorporated in the composition without affecting the germ killing property.
b. Food grade organic or inorganic acids are present as acidifying agents to lower the pH at or below 7.0. The acid is selected from adipic acid, ascorbic acid, benzoic acid, citric acid, dehydroacetic acid, erythorbic acid, fumaric acid, glutaric acid, gluconic acid, hyaluronic acid, hydroxyacetic acid, lactic acid, malic acid, salicylic acid, sorbic acid, succinic acid, suberic acid, azelaic acid, sebacic acid, tannic acid, tartaric acid, carboxylic acid polymers, homo- or hetero-polymerized carboxylic acid such as poly lactic acid or poly lactic-glycolic acid; or mixtures thereof.
c. Suitable solubilizing agent is selected from water and/or various nontoxic alcohols or glycols or a mixture thereof.

The biologically active sanitizing liquid spray/inhaler composition is based on (a) cleaning, surface active properties of anionic or surface active agent or agents, (b) microbicidal properties of either anionic surface active agent under acidic conditions or a mixture of organic acids, (b) the organic/inorganic acids as acidifying agents or their mixture as microbicidal agents (c) water or suitable solubilizing agent such nontoxic alcohols, glycols or a mixture. Additional ingredients can be incorporated to fortify the microbicidal properties these include tuberculocidal and cysticidal activities of esters of fatty acids; and/or (e) fungicidal properties of benzoic acid and its salts or esters.

The anionic surface-active agent is selected from either free acid or salt forms the following classes of compounds:
(a) C6-C18 alkyl- and alkenyl-sulfates; (b) C6-C18 alkyl- and alkenyl-ether sulfates; (c) C8-C16 alkyl diphenyl ether disulfonates; (d) C4-C18 fatty acid isethionates; (e) C6-C18 alkyl- and alkenyl sulfonates; (f) dialkyl- and dialkenyl sulfosuccinates in which the alkyl or alkenyl groups independently contain from six to eighteen carbon atoms; (g) C6-C18 alkylbenzene sulfonates; (h) naphthalene sulfonates; (i) alkyl naphthalene sulfonates in which alkyl group contains from one to six carbon atoms; (j) the mono-(n-alkyl) and mono-(n-alkenyl) acyl esters of C2-C4 hydroxylated monocarboxylic acids in which the alkyl or alkenyl group contains from six to eighteen carbon atoms; (k) the mono-(n-alkyl) and mono-(n-alkenyl) acyl esters of C2-C4 hydroxylated dicarboxylic acids in which the alkyl or alkenyl group contains from six to eighteen carbon atoms; (l) the mono-(n-alkyl) and mono-(n-alkenyl) alkyl esters of C2-C4 dicarboxylic acids, in which the alkyl or alkenyl group contains from six to eighteen carbon atoms; and (m) C4-C18 fatty alcohol sulfoacetates.

The anionic agent used in the nasal and sinus cleaning and disinfecting composition is preferably sodium dodecyl sulfate, sodium dioctyl sulfosuccinate, sodium decyl lactylate, sodium dodecylbenzene sulfonate, and sodium alpha-olefin sulfonate and a mixture thereof. The concentration of anionic agent/agents preferably is between 0.0010% w/v to 15.0% w/v. As used herein, a 1% solution would have 1 gram of solute dissolved in a final volume of 100 milliliters of solution. In this disclosure, such units are labeled as weight/volume (w/v) percentage solution.

The acidifying agent is used to lower the pH below 7.0, preferably at or around pH 3.0. The acidifying agent used to lower the pH must be compatible with the anionic surface-active agent. The acidifying agent is one or more of adipic acid, ascorbic acid, citric acid, dehydroacetic acid, erythorbic acid, fumaric acid, glutaric acid, gluconic acid, hyaluronic acid, hydroxyacetic acid, lactic acid, malic acid, succinic acid, tannic acid, tartaric acid, adipic acidpimelic acid, suberic acid. azelaic acid. sebacic acid, carboxylic acid polymers, homo- or hetero-polymerized carboxylic acid such as poly lactic acid or poly lactic- glycolic acid; or mixtures thereof.

The preferred acidifying agent is one or more selected from citric acid, benzoic acid, sorbic acid, adipic acid, or lactic acid or a mixture thereof used at concentrations of 0.01% w/v to 10% w/v.

The solubilizing agents can be selected water and/or non-toxic alcohols and glycols. The preferred solubilizer used is deionized or distilled water to complete 100% w/v of the composition.

The composition optionally contains sodium chloride at 0.5% w/v to 5% w/v. The other optional ingredients could include color and fragrances. The emollient agent used is the powdered extract of aloe vera used at concentrations between 0.05% w/v to 2% w/v.

The cleaning and disinfecting solution optionally contains natural antimicrobial and medicinal herbs and their ingredients, extracts and/or oils and vitamins such A, C, D and E, tea tree oil fatty alcohols, fatty alcohol esters, sphingolipids, thyme, menthol, ginger, turmeric, eugenol, rosemary, eucalyptol, thymol, coriander, fenugreek, tulsi, basil, lemongrass alone or mixture thereof at concentrations of 0.01% w/v to 5.0% w/v.

The cleaning and disinfecting solution optionally contains antiviral agents such as derivatives or analogs of n-aceryl neuraminic acid, Amantadine, Arbidol, Oseltamivir, Peramivir, Rimantadine, Zanamivir, Abacavir, Didanosine, Emtricitabine, Lamivudine, Stavudine, Zalcitabine, Zidovudine, Tenofovir, Efavirenz, Delavirdine, Nevirapine, Loviride Amprenavir, Atazanavir, Darunavir, Fosamprenavir, Indinavir, Lopinavir, Nelfinavir, Ritonavir, Saquinavir, Tipranavir, Enfuvirtide, Adefovir, Fomivirsen, Imiquimod, Inosine, Interferon, Podophyllotoxin, Ribavirin, Viramidine, alone or mixture thereof at concentrations of 0.0001% w/v to 5.0% w/v.

The cleaning and disinfecting solution optionally contains decongestant such as Ephedrine, Oxymetazoline, Phenylephrine, Pseudoephedrine, Tramazoline, Xylometazoline alone or mixture thereof at concentrations of 0.0001% w/v to 5.0% w/v.

The cleaning and disinfecting composition optionally contains antihistaminic and antiallergic compounds to control allergic conditions.

The cleaning and disinfecting composition optionally contains gums and polymers for slow release of the disinfecting properties. The gums and the polymers are at concentrations from 0.001% w/v to 10.0% w/v.

The cleaning and disinfecting composition for nasal and sinus cavities is be prepared either as solution spray for instillation or as cream for coating nostrils prepared using thickening and solidifying agents.

### The Nasal and Sinus Cleaning and Disinfecting Compositions and Their Efficacy

The examples in Table 1 show the preparation of wipe sanitizers using anionic surface-active agent and an acidifying agent. The compositions are prepared to a final 100% w/v with deionized water.

To ascertain the sanitizing efficacy and microbicidal spectrum against bacteria, three sanitizers were first evaluated by the sanitizer and germicidal detergent test specified by the Association of Analytical Chemists (A.O.A.C.), 1984, using Escherichia coli ATCC 11229 and Staphylococcus aureus ATCC 6538. The bacterial cultures were purchased from the American Type Culture Collection (ATCC), (Bethesda, Md.). Escherichia coli and Staphylococcus aureus are used to represent both gram-negative and gram-positive bacterial classes. This test is used by the US EPA for to determine compliance for a product to be used as a sanitizing agent. A product that reduces population density of both types of bacteria in 30 seconds by 99.999% satisfies the requirement as a sanitizing agent. Fifty ppm hypochlorite meets the criterion of a sanitizer in this test against Escherichia coli and Staphylococcus aureus and was used as a positive control.

The efficacy of the sanitizers in Examples 1, 2 and 3 was further evaluated with 50 of hypochlorite as controls against Salmonella typhimurium, Listeria monocytogenes, Aeromonas hydrophila, and Pseudomonas aeruginosa essentially by the modified detergent and germicidal sanitizer test of the A.O.A.C. (Lopes, 1986) as shown in Table 2. The sanitizers were tested against both natural isolates and the antibiotic resistant strains of Listeria monocytogenes and Salmonella typhimurium. Each of the antibiotic resistant strains was used separately as well as in a composite strain prepared by mixing equal numbers of five different antibiotic resistant strains of Listeria monocytogenes or those of Salmonella typhimurium, respectively.

### The Nasal and Sinus Cleaning and Disinfecting Composition and Efficacy Against Bacteria

TABLE 1
Examples of nasal and sinus cleaning and disinfecting compositions with
anionic surface-active agent and acidifying agent.

| | | | % Kill | | |
|---|---|---|---|---|---|
| Sanitizing | | | | Staph. | |
| Composition | Ingredients | % w/v | | aureus | E. coli |
| Example 1 | Citric acid | 0.37 | | >99.999 | >99.999 |
| | Sodium dodecyl sulfate | 0.028 | | | |
| Example 2 | Acetic acid | 0.97 | | >99.999 | >99.999 |
| | Dioctyl Sulfosuccinate | 0.029 | | | |
| Example 3 | Lactic Acid | 0.85 | | >99.999 | >99.999 |
| | Decyl Lactylate | 0.029 | | | |
| Control | Hypochlorite | 50 | ppm | >99.999 | >99.999 |

TABLE 2
Efficacy of sanitizers against selected microorganisms by the A.O.A.C.
germicidal and detergent sanitizer test.

| | | Number of surviving | | | |
|---|---|---|---|---|---|
| | | bacteria/ml after contact | | | |
| | | 30 seconds | | 60 seconds | |
| Test organism | Test Sanitizer | cfu/ml | % Kill | cfu/ml | % Kill |
| A. h. 7965 | Example 1 | 0, 0 | >99.999 | 0, 0 | >99.999 |
| L. m. 7644 | | 2, 3 | >99.999 | 0, 0 | >99.999 |
| P. a. 10145 | | 0, 0 | >99.999 | 0, 0 | >99.999 |
| S. t. 7823 | | 0, 0 | >99.999 | 0, 0 | >99.999 |
| A. h. 7965 | Example 2 | 4, 2 | >99.999 | 0, 0 | >99.999 |
| L. m. 7644 | | 0, 0 | >99.999 | 0, 0 | >99.999 |
| P. a. 10145 | | 0, 0 | >99.999 | 0, 0 | >99.999 |
| S. t. 7823 | | 1, 0 | >99.999 | 0, 0 | >99.999 |
| A. h. 7965 | Example 3 | 0, 0 | >99.999 | 0, 0 | >99.999 |
| L. m. 7644 | | 0, 0 | >99.999 | 0, 0 | >99.999 |
| P. a. 10145 | | 7, 1 | >99.999 | 1, 2 | >99.999 |
| S. t. 7823 | | | >99.999 | 0, 0 | >99.999 |
| A. h. 7965 | Hypochlorite | 1*, 0* | >99.999 | 0, 0 | >99.999 |
| L. m. 7644 | | 82*, 60* | >99.999 | 17, 16 | >99.999 |
| P. a. 10145 | | 1, 2 | >99.999 | 1, 0 | >99.999 |
| S. t. 7823 | | T@, T@ | <99.999 | 86, 94 | >99.999 |

*HOCL = 200 ppm. @ HOCL = 50 ppm. T = Too Numerous To Count. A. h. = Aeromonas hydrophila, L. m. = Listeria monocytogenes, P. a. = Pseudomonas aeruginosa, S. t. = Salmonella typhimurium.

### Tuberculocidal Activity of the Cleaning and Disinfecting Composition

To be more useful in environment where mycobacteria might be present, the nasal and sinus cleaning and disinfecting composition can also be prepared with tuberculocidal activity. Decyl lactylate was incorporated in the sanitizing and microbicidal wipe composition to impart tuberculocidal activity. An aqueous solution of 300 ppm of decyl lactylate and 8800 ppm of lactic acid (pH ≦3.0) exhibited higher tuberculocidal activity than 100 ppm of hypochlorite when examined essentially by the AOAC germicidal and detergent sanitizer test (Table 2). The tests were carried out in 500 ppm of hard water prepared according to the AOAC procedure (AOAC, 1990). The neutralizing solution used consisted of lecithin with polysorbate 80 prepared in phosphate buffer pH 7.2. In case of hypochlorite 0.1 ml of 10% thiosulfate was added to each tube of the neutralizer solution.

The culture was prepared according to the AOAC procedure for testing tuberculocidal activity of disinfectants (AOAC, 1990). Mycobacterium tuberculosis H37 Ra was maintained on Middlebrook 7H9 agar consisting of 4.7 g Middlebrook 7H9 broth (Difco), 2 ml of glycerol (Sigma) and 15 g Bacto-Agar in 900 ml of deionized water and 100 ml of Middlebrook ADC enrichment (Difco). The same medium was used for surface plating during the test. To provide aerobic conditions for Mycobacterium tuberculosis surface plating was used on pre-poured plates. Pre-poured plates were dried by storing at room temperature for one week prior to use. The culture from a 20-day-old slant was inoculated into modified Proskauer-Beck broth and incubated undisturbed at 37° C. for 25 days. The culture from a single tube was homogenized to a smooth suspension free of visible clumps with 1 ml of 0.1% Tween 80 in 0.9% NaCl. The culture was diluted to give 20% transmission at 650 nm and used in the test.

A germicidal and detergent sanitizer test (AOAC, 1990) was essentially employed for evaluating tuberculocidal of decyl lactylate. Ninety-nine ml of the test sanitizer solution contained 300 ppm of decyl lactylate and 8800 ppm of lactic acid. The test was carried out by rapidly mixing 1 ml of the test suspension into 99 ml of the sanitizer solution. After contact period of 30 seconds, 60 seconds, and 5 minutes, one ml of the test mixture was removed and added to 9 ml of the neutralizer. Clorox at 100 ppm was used as positive control. One ml of the neutralized test mixture was plated on Middlebrook 7H9 agar. The culture was diluted ten fold with 0.02% Tween 80 and plated to estimate the number of bacteria used in the test. The plates were replaced in the original plastic plate bags, which were sealed with adhesive tape to prevent drying and incubated at 37° 2 C. for three weeks. The colonies were counted and reduction in colony count compared to controls was used to calculate efficacy of the sanitizers.

TABLE 3
Lethal activity of nasal and sinus cleaning and disinfecting composition
against Mycobacterium tuberculosis.

| | CFU After Contact | | |
|---|---|---|---|
| | Time in Seconds/minutes | | |
| | 30 Seconds | 60 Seconds | 5 Minutes |
| Example 3 | % Kill | % Kill | % Kill |
| Decyl lactylate | ≧99.996 | ≧99.999 | ≧99.999 |
| (0.029%) | ≧99.996 | ≧99.999 | ≧99.999 |
| NaOCl (100 ppm) | ND | ND | ≧99.998 |
| | ND | ND | ≧99.997 |

Activity of decyl lactylate under acidic pH (<3.0);
CFU = colony forming unit,
ND = Not determined,
T = Too Numerous To Count (TNTC),
Challenge Number of CFUs/ml = 72 × 10⁶

The examples show microbicidal efficacy of sanitizing wipe compositions against gram positive, gram negative as well as tuberculosis bacteria. The tests show that the sanitizing composition has a broad-spectrum lethal activity against microorganisms. Thus the lethal activity of the nasal and sinus cleaning and disinfecting composition can be used to disinfect mucosal surfaces contaminated with bacteria, fungi, protozoa and viruses.

The examples presented above are merely illustrative and should not be read as limiting the scope of the invention as it is defined in the appended claims.

While embodiments of the invention have been illustrated and described, it is not intended that these embodiments illustrate and describe all possible forms of the invention. Rather, the words used in the specification are words of description rather than limitation, and it is understood that various changes may be made without departing from the spirit and scope of the invention.

What is claimed is:

1. A biologically active composition for cleaning and disinfecting a nasal tract and a sinus cavity, comprising:
one or more organic acidifying agents;
one or more inorganic acidifying agents;
one or more anionic surface-active agents; and
one or more solubilizing agents.

2. The biologically active composition in claim 1, wherein the composition has a formulation that is selected from the group consisting of a solution, a spray and a mist.

3. The biologically active composition in claim 1, wherein the composition comprises a cream.

4. The biologically active composition in claim 1, wherein the acidifying agents have a pH which is less than or equal to 7.0.

5. The biologically active composition in claim 1, wherein the one or more organic acidifying agents include an acid selected from the group consisting of: adipic acid, ascorbic acid, benzoic acid, citric acid, dehydroacetic acid, erythorbic acid, fumaric acid, glutaric acid, gluconic acid, hyaluronic acid, hydroxyacetic acid, lactic acid, malic acid, succinic acid, tannic acid, tartaric acid, pimelic acid, suberic acid, azelaic acid, sebacic acid, carboxylic acid polymers, homo- or hetero-polymerized carboxylic acid such as poly lactic acid, polylactic glycolic acid; and mixtures thereof.

6. The biologically active composition in claim 1, wherein the one or more organic acidifying agents includes an agent selected from a group consisting of benzoic acid, citric acid, lactic acid, sorbic acid, adipic acid and mixtures thereof.

7. The biologically active composition in claim 1, wherein one or more of the organic acidifying agents comprises a food grade agent.

8. The biologically active composition in claim 1, wherein the formulation comprises one or more inorganic acidifying agents.

9. The biologically active composition in claim 1, wherein one or more of the inorganic acidifying agents comprises a food grade agent.

10. The biologically active composition in claim 1, wherein the one or more anionic surface-active agents include an agent selected from the group consisting of:
a. C6-C18 alkyl- and alkenyl-sulfates;
b. C6-C18 alkyl- and alkenyl-ether sulfates;
c. C8-C16 alkyl diphenyl ether disulfonates;
d. C4-C18 fatty acid isothionates;
e. C6-C18 alkyl- and alkenyl sulfonates;
f. dialkyl- and dialkenyl sulfosuccinates in which the alkyl or alkenyl groups independantly contain from six to eighteen carbon atoms;
g. C6-C18 alkylbenzene sulfonates;
h. naphthalene sulfonates;
i. alkyl naphthalene sulfonates in which alkyl group comprised of one to six carbon atoms;
j. the mono-(n-alkyl) and mono-(n-alkenyl) acyl esters of C2-C4 hydroxylated monocarboxylic acids in which the alkyl or alkenyl group comprised of from six to eighteen carbon atoms;
k. the mono-(n-alkyl) and mono-(n-alkenyl) acyl esters of C2-C4 hydroxylated dicarboxylic acids in whicht eh alkyl or alkenyl group comprised of six to eighteen carbon atoms;
l. the mono-(n-alkyl) and mono-(n-alkenyl) alkyl esters of C2-C4 hydroxylated dicarboxylic acids in whicht eh alkyl or alkenyl group comprised of six to eighteen carbon atoms; and
m. C4-C18 fatty alcohol sulfoacetates; and
n. mixtures thereof.

11. The biologically active composition in claim 10, further comprising anionic surface-active agent concentrations ranging from 0.001% w/v to 15% w/v.

12. The biologically active composition in claim 10, wherein one or more of the anionic surface-active agents are selected from the group consisting of: sodium dodecyl sulfate, sodium docyl lactylate, sodium dioctyl sulfosuccinate, sodium dodecylbenzene sulfonate, sodium alpha-olefin sulfonate, and mixtures thereof.

13. The biologically active composition in claim 9, wherein one or more anionic surface-active agents has a form that comprises a free acid.

14. The biologically active composition in claim 9, wherein one or more anionic surface-active agents is a has a form that comprises a salt.

15. A biologically active composition in claim 1, further comprising:
one or more medicinal emollients, soothing agents, antihistiminic agents, antiallergic agents, synthetic chemicals, pharmaceutically active ingredients and natural medicinal herbs selected from the group consisting of:
vitamins (A, C, D, and Q);
aloe vera;
tree oil;
fatty alcohols;
fatty alcohol esters;
sphingolipids;
benzoic acid;
antivirals;
decongestants;
thyme;
menthol;
ginger;
tumeric;
eugenol;
rosemary;
perilla;
eucalyptol;
thymol;
coriander;
fenugreek;
tulsi;
basil;
lemongrass; and
extracts, oils and mixtures thereof.

16. The biologically active composition in claim 14, wherein medicinal emollients, synthesized chemicals, pharmacologically active ingredients medicinal herbs and soothing agents are present in concentrations between 0.0001% w/w to 5.0% w/w.

17. The biologically active composition in claim 15, further comprising one or more gums and one or more polymers for slow release of disinfecting properties.

18. The biologically active composition in claim 1, wherein the one or more solubilizing agents contain propylene glycol.

19. The biologically active composition in claim 1, wherein the one or more solubilizing agents comprise a food grade solvent selected from the group consisting of: water, nontoxic alcohols, glycols and mixtures thereof.

20. The biologically active composition in claim 1, further including one or more food additives.
